(19) United States
(12) Patent Application Publication
(10) Pub. No.: US 2026/0061214 A1
(43) Pub. Date: Mar. 5, 2026

(54) TMS Coil and TMS System

(71) Applicant: Rheinland Pfaelzische Technische Universitat Kaiserslautern-Landau, Kaiserslautern (DE)

(72) Inventors: Stefan M. Götz, Kaiserslautern (DE); Max Köhler, Kaiserslautern (DE)

(21) Appl. No.: 19/106,067

(22) PCT Filed: Aug. 21, 2023
(86) PCT No.: PCT/EP2023/072938

§ 371 Date: Feb. 24, 2025

(30) Foreign Application Priority Data
Aug. 24, 2022 (DE) 10 2022 121 390.0

(51) Int. Cl. A61N 2/02; A61N 2/00

(52) U.S. Cl. CPC A61N 2/02; A61N 2/006

(57) ABSTRACT

A coil for the transcranial magnetic stimulation of a human brain, is designed and set up to generate an alternating magnetic field, with: at least one electrical line with a first end and a second end, at each of which a feed connection for supplying the coil with electrical energy is arranged, and which has at least four windings with a different distance from the adjacent winding at least in sections; and an insulating structure of a non-conductive material for abutment against a head of a person to be treated.

### TECHNICAL FIELD

The present invention relates to a coil for transcranial magnetic stimulation of a human brain and a corresponding system comprising such a coil and a corresponding method.

### BACKGROUND

Transcranial magnetic stimulation is a non-invasive treatment method for the human brain and is used for various treatment purposes. For example, it can be used to effectively treat affective disorders, in particular clinical depression, obsessive-compulsive disorder or post-traumatic stress disorder.

The mode of action of transcranial magnetic stimulation is essentially based on the fact that neurons and neuronal circuits of defined brain areas, which can themselves comprise several neurons and are also referred to as neuronal excitation circuits, are stimulated from the outside by means of an externally generated alternating (electro)magnetic field, so that the stimulated neutrons in turn emit signals. The resulting signals are transmitted and processed by other neurons and neuronal circuits in the brain. By using defined parameters to generate the alternating magnetic field for stimulating the neurons, neurons and neuronal circuits can be neuromodulated as required. As a result, neuromodulated neurons and neuronal circuits are able to process endogenous signals in particular, i.e. the body's own signals, in the long term and beyond the duration of treatment. This has a positive effect on the therapeutic treatment of the above-mentioned disorders. A significant advantage is that treatment using transcranial magnetic stimulation does not require the additional intake of psychotropic drugs, which are known to have negative effects on human well-being. Transcranial magnetic stimulation is the subject of extensive research, particularly with regard to the biophysical modeling of neurostimulation and the spatial-physiological modeling of the areas of the brain to be stimulated. For the technological background of the invention, reference is made to the scientific article by Goetz & Deng (2017) “The development and modeling of devices and paradigms for transcranial stimulation”, International Review of Psychiatry, 29:2, 115-145 [doi: 10.1080/09540261.2017.1305949], the contents of which are made part of the disclosure of this application.

Coils with several windings are used to generate the alternating magnetic field in a known manner. However, one challenge is to design the coil in such a way that the field characteristics of the alternating magnetic field are optimally adapted to the areas of the human brain to be stimulated. Known coils therefore have windings with complex, partially overlapping geometries, so that locally very high field strengths can be generated at other, undesired points, which can harm the patient, or at least represent a side effect for the patient. In addition, the known coils have to be cooled at great expense due to the high field strengths. Finally, the known coils are also expensive due to the complex winding geometries.

In particular, special coils are used to stimulate the cortical surface of the brain. Some are intended, for example, for focal points in the primary motor cortex or the dorsolateral prefrontal cortex and accordingly generate very concentrated, focal electric fields at this or these points and low electric field strengths outside, other coils activate neurons in a large, well-defined cortical area, which includes the DLPFC, for example, and have correspondingly more extensive areas with high electric field strengths, possibly also with a specific electric field direction in each case. What they all have in common is that the irritated area is well-defined with a certain minimum electric field strength and that a certain electric field strength is not exceeded in other areas for minor side effects. Certain (neuro-) physiological effects and the corresponding procedures for their generation are often linked to a certain spatial field distribution, which can be generated by different coils, but whose field distribution in the brain is sufficiently similar.

Against this background, a person skilled in the art is faced with the task of creating a coil, in particular a TMS coil, and a system and method by means of which the performance of transcranial magnetic stimulation can be improved and, in particular, simplified in terms of handling. A cost-efficient way of creating a TMS coil that is suitable for cost-efficient operation is particularly preferred.

### SUMMARY

The above task is solved by a coil for the transcranial magnetic stimulation of a human brain, which is designed and set up to generate an alternating magnetic field, with:
at least one electrical line with a first end and a second end, at each of which a feed connection for supplying the coil with electrical energy is arranged, and which has at least four windings with at least partially different spacing from the adjacent winding; and
an insulating structure made of a non-conductive material for contact with a head of a person to be treated, which is arranged below the windings of the electrical line in such a way that the insulating structure electrically insulates the line from the head of a person to be treated, wherein
the coil comprises two groups of windings;
a first group comprises inner windings;
comprises a second group of outer windings; and
a first surface is formed by a first surface enclosing the first group of turns; and a second surface is formed by a second surface enclosing the second group of turns; and
the second area is at least 1.5 times as large as the first area.

The above problem is further solved by a system for transcranial magnetic stimulation of a human brain, comprising:
a coil as defined above; and
a control unit for controlling the coil in order to form an alternating magnetic field by means of the coil.

Finally, the above problem is solved by a method for transcranial magnetic stimulation of a human brain by means of a coil as defined above or by means of a system as defined above, comprising the steps:
arranging the insulating structure of the coil in an environment of a head of a person to be treated; and
Formation of an alternating magnetic field for transcranial magnetic stimulation of the brain of the person to be treated by activating the coil.

An electrical conductor with at least four windings, at least sections of which have a different distance from the neighboring winding, can be used to create a coil with an advantageous geometry in a technically simple manner. Distances between windings according to the invention can, for example, denote gaps between windings, but also distances between centers of the cross-sections of the corresponding windings. An insulating structure enables increased safety during treatment with such a coil, since energy input in the form of electrical energy from the coil to the person to be treated can be prevented. In particular, the insulating structure can be a layer of lacquer arranged around the windings of the coil. In other words, the coil may be lacquered. It is understood that the coil can also be encapsulated with an insulator, for example a resin, or wrapped with an insulator. In particular, the insulating structure can also take the form of a plate and be arranged only on the side of the coil facing the head of the person to be treated during treatment.

Despite the simple geometry with only at least four windings, two groups of windings make it possible to generate a magnetic field comparable with known TMS coils. In particular, comparable or better stimulation results can be achieved. It has been shown that a coil with such a structure can stimulate individual regions of the human brain in a more targeted manner. Due to the advantageous design, production costs and cooling costs for such a coil can be reduced.

By means of a second surface which is at least 1.5 times as large as the first surface, a flat coil with a geometry adapted to the stimulation of a human brain can be created with respect to the alternating magnetic field formed by the coil, in a departure from previous concepts. Due to the geometry, a coil can be created which is easy to handle, can be manufactured cost-effectively and is multifunctional, i.e. can be used by different people. In particular, a coil can be created that can be operated in an energy-efficient manner.

An enclosure surrounding the windings is preferably to be understood as a closed surface in which the windings are located, in particular as a smallest surface that is largely in contact with the winding at its boundary. In particular, the enclosing end can be understood as the smallest surface that completely covers the winding. For example, the enclosing end can be formed by directly connecting two ends of a winding.

A weight-optimized TMS coil with improved handling can be created by using two groups of windings whose areas form a ratio of 1 to at least 1.5 and which, at least in sections, have a different distance to the respective adjacent winding. The advantageous interaction of the area ratio and variable spacing configuration can generate an alternating magnetic field that is suitable for transcranial magnetic stimulation. Passive cooling of the TMS coil is preferably possible, which contributes to improved handling as well as lower operating and production costs. In addition, passive cooling enables a wider range of applications.

In a preferred embodiment, the coil is formed with a curvature and is curved in one dimension on average with less than 2π/3 rad (120°) arc length, preferably less than π/2 rad (90°) arc length and particularly preferably less than π/2 rad (60°) arc length, relative to a center of the curvature or in two dimensions on average with a solid angle of less than π sr (steradian), preferably of less than ⅔ π sr and particularly preferably of less than π/2 sr. In addition, a radius of curvature of the curvature is at least 60 mm, preferably at least 85 mm and particularly preferably at least 110 mm. Despite its simple geometry, the curvature of the coil allows an advantageous input of the alternating magnetic field into the brain for stimulation. In particular, this makes it possible to create a coil that can be manufactured efficiently and cost-effectively and allows simplified handling. A curvature makes it easier to position the coil on the head of a person to be treated. In particular, the described curvature can be used to find a compromise between the manufacturability of the coil and the achievable field strength.

The windings of the coil are preferably arranged planar. This makes it possible to create a technically efficient and cost-effective coil. A coil can be created that allows easy handling and yet precise alignment. Preferably, such a coil can be suitable for stimulation in which the coil is guided as a hand-held device.

In particular, it is conceivable that individual groups of windings of the coil are each arranged planar, in particular in one plane, whereby the individual planes can be arranged offset to each other. One of these planes can, for example, be spaced up to 10 mm in the direction of the normal in both directions from another of these planes.

Advantageously, the windings of the electrical cable between the feed connections are arranged without kinks. Additionally or alternatively, at least one winding has a negative curvature in at least one area. In particular, kink-free means that a function describing the course of the windings between the feed connections is continuously differentiable everywhere. Negative curvature means that a tangent to a function describing the winding intersects the winding or the function. A negative curvature or a negative radius of curvature means in particular that a tangent to the winding of the electrical line would intersect this winding of the electrical line. If the winding does not have a negative radius of curvature, no tangent can be found for this winding that intersects this winding. Bend-free windings can increase the mechanical stability of the coil. Particularly with hand-guided coils, damage to the coil in the event of accidental dropping or bumping can be counteracted. At least one area in which the winding has a negative curvature can be used to create a coil with a highly variable geometry that can be optimized to generate the desired alternating magnetic field. In particular, this scope in the geometry enables the creation of a compact coil with sufficiently high field strength and field geometry of the alternating magnetic field.

Advantageously, the electrical line comprises between four and twenty windings. Additionally or alternatively, the coil an inductance in the range from 4 μH to 5 mH, preferably in the range from 5 μH to 500 μH and particularly preferably in the range from 5 μH to 30 μH. In addition or alternatively, the coil has an electrical resistance in the range from 1 mΩ to max. 1,000 mΩ, preferably in the range from 2.5 mΩ to max. 750 mΩ and particularly preferably in the range from 5 mΩ to 50 mΩ. The ranges claimed above for the windings, the electrical resistance and the inductance can be used to create a highly variable coil that can be optimally adapted to a specific application. In particular, a coil can be created that can be operated in an energy-efficient manner and preferably requires less cooling. Preferably, a coil can be created that is cost-efficient to manufacture and cost-efficient to operate.

Particularly preferably, the second area is at least 1.75 times, preferably at least 2 times and particularly preferably at least 2.5 times, as large as the first area. These advantageous ratios allow the alternating magnetic field and a magnetic vector potential to be made more precise. In particular, an area of the human brain can be stimulated more precisely without stimulating areas that are not to be stimulated.

Advantageously, the coil has exactly one single electrical line that forms the windings. The windings are preferably arranged without overlapping. This further simplifies the manufacture of the coil. Because the individual conductor paths do not overlap, the interaction between the individual conductor paths can be reduced and the efficiency of the coil can be increased. Furthermore, a coil can be created that can be manufactured quickly and cost-effectively.

In a further advantageous embodiment, the windings have at least two different centers. In particular, the center point is understood to be the point with the smallest distance to all sections of a winding. In particular, the center point can also be understood as the center of gravity of a winding. A winding is preferably to be understood as a 360° turn of the electrical cable. It is understood that the cable does not necessarily have to form a circle, but can assume other geometric shapes. Consequently, only one conductor track is arranged in each direction when viewed from the center of the winding. Preferably, the first group has a center point that is different from the second group, whereby the center points of the individual windings of a group are essentially the same in each case. In particular, different centers enable the formation of an asymmetrical magnetic field so that stimulation can be more precisely localized.

Preferably, the centers of the at least two surfaces are at least 20 mm, preferably at least 30 mm and particularly preferably at least 40 mm apart. Several windings forming a first surface can also have a common center point, while at the same time several windings forming a second surface can also form a common center point.

Further preferably, the windings of the electrical line have a direction-dependent asymmetry, with a first asymmetry with respect to a first direction in the coil plane, which preferably comprises different conductor densities of the electrical line, and a second asymmetry with respect to a second direction running perpendicular to the first direction in the coil plane, which preferably comprises different bending radii of the electrical line. The coil plane is to be understood in particular as the plane or surface in which the windings of the electrical cable are located. It is understood that this can be understood to mean a curved and/or curved plane or curved surface. A direction-dependent asymmetry allows the alternating magnetic field formed to be made more precise so that high-precision stimulation can be achieved using the coil. In particular, the asymmetries in different directions, preferably perpendicular to each other, enable a highly precise determination and formation of the magnetic field.

The control unit can comprise a voltage source and/or a current source and, in particular, have two connections via which the coil can be supplied with electrical energy. In particular, the control unit can have a function generator, which is designed in particular to generate periodic electrical signals with user-defined parameters such as signal shape, pulse duration, frequency or amplitude in order to generate a desired alternating magnetic field. The control unit can be designed to control the coil in particular with current pulses that have a pulse duration of maximum 5 ms, in particular maximum 1 ms, for example between 50 μs and 350 μs, preferably between 10 μs and 1000 μs, in particular between 120 μs and 400 μs, so that the field characteristics of the magnetic field to be generated can be optimized. The control unit can be designed so that a magnetic flux density of at least 1 T and/or an electric field strength of at least 10 V/m, in particular at least 50 V/m, preferably at least 100 V/m can be generated by the coil. The control unit can be designed to apply a voltage of at least 100 V, in particular a variable voltage of at least 1000 V, to the coil.

The electrical conductor that forms the windings of the coil can have different cross-sections in sections. Furthermore, several conductor sections can comprise different materials, for example copper, aluminum or other metals or electrical conductors. In particular, it is conceivable that one section is punched, water jet-cut or laser-cut and another section is produced using a different manufacturing technique. These sections are then joined together to form the electrical cable of the coil.

Due to the advantageous geometry of the coil, there are points from which the current flows in the same direction of rotation.

Windings can be formed that not only have an inward curvature, but at times or in sections also have an outward curvature.

In particular, the different geometries of the individual windings can form indentations or lagoons, i.e. conductor-free areas surrounded by conductors, at least in sections. A vector describing the direction of one or both of the surrounding conductors can therefore have a double sign change in the second path derivative. In particular, the respective current of both conductors surrounding an indentation or lagoon points in the same direction. A current pointing in the same direction means, in particular, that there is at least one point in each of the conductors surrounding an indentation or lagoon at which a vector describing the current flow points in the same direction.

Preferably, there are at least two clusters or groups of windings, with the windings in the groups each spanning similar areas to each other and representatives from different groups spanning different areas.

The windings of the coil can be arranged in such a way that at least two surface areas are formed which are surrounded by windings on all sides, whereby a first surface area is surrounded on at least two opposite sides by windings whose respective currents point in the same direction, and a second surface area is surrounded on at least two opposite sides by windings whose respective currents point in opposite directions. Preferably, the first surface area has windings on two further opposite sides, whose currents point in opposite directions. These two surface areas preferably do not overlap and/or are also preferably not intersected by any windings. Preferably, one surface area, preferably each of the surface areas, comprises at least one tenth, particularly preferably at least one seventh of the total area of the coil enclosed by the outermost winding. By sides of windings whose respective current points in the same (opposite) direction, it is to be understood in particular that at least one point is present in each of the opposite windings enclosing the surface area, in which a vector describing the current flow points in the same (opposite) direction.

### BRIEF DESCRIPTION OF THE DRAWING FIGURES

The invention is described and explained in more detail below with reference to some selected embodiments in connection with the accompanying drawings. It shows:

FIG. 1 a schematic simplified representation of a coil for transcranial magnetic stimulation in a plan view;

FIG. 2 a section through the coil according to FIG. 1 along the sectional plane A;

FIG. 3 a system for transcranial magnetic stimulation of a human brain comprising a coil as shown in FIGS. 1 and 2 and a control unit;

FIGS. 4a to 4i Diagrams of different coil geometries for coils according to the invention coils for transcranial magnetic stimulation according to the invention in a top view;

FIG. 5 schematic of a simulated magnetic field strength during magnetic stimulation by means of a coil according to the invention; and

FIG. 6 schematic of the steps of a method according to the invention for transcranial magnetic stimulation of a human brain.

### DETAILED DESCRIPTION

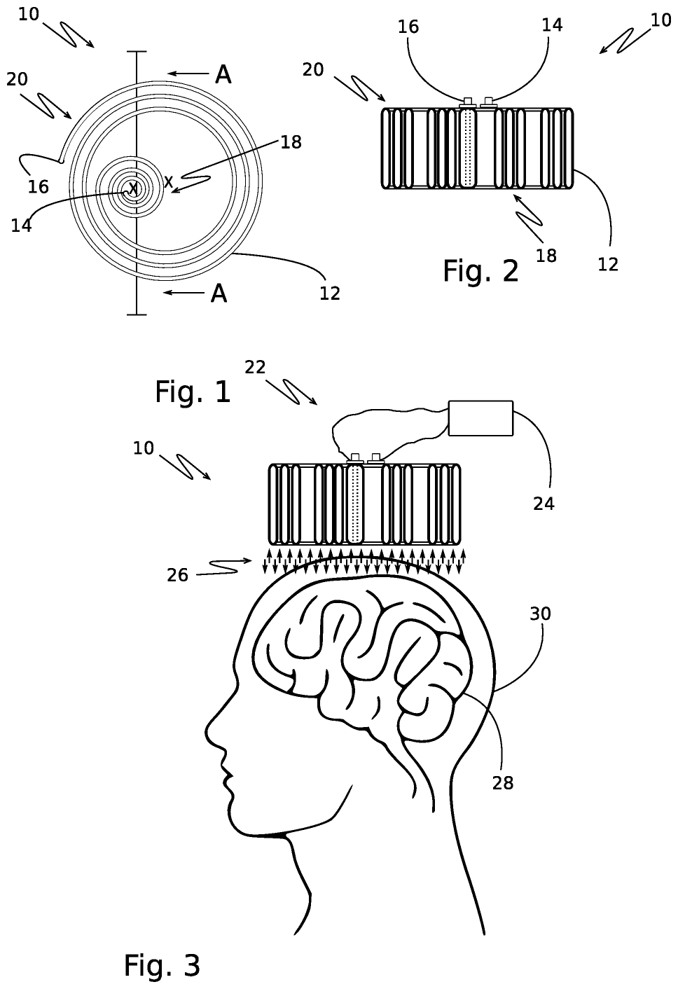

FIG. 1 shows a simplified schematic diagram of a coil 10 for transcranial magnetic stimulation (TMS) of a human brain. The coil 10 comprises an electrical cable 12 that forms several windings. Feed connections 14, 16 are arranged at the two ends of the electrical line 12 in order to connect the coil 10 to a control device, not shown, so that the coil 10 can be controlled. The electrical cable 12 forms a first group 18 of inner windings, i.e. windings with smaller bending radii, and a second group 20 of outer windings, i.e. windings with larger bending radii.

The first group 18 approximately encloses an area that is at least 1.5 times smaller than the area enclosed by the second group 20. An enclosing end of the second group 20 is therefore 1.5 times as large as an enclosing end of the first group 18.

Furthermore, the first group 18 and the second group 20 each have different center points, whereby the center points for windings of a group are essentially the same in the example shown and are each shown as “X” in the figure.

FIG. 1 also shows a sectional plane A with the direction of view.

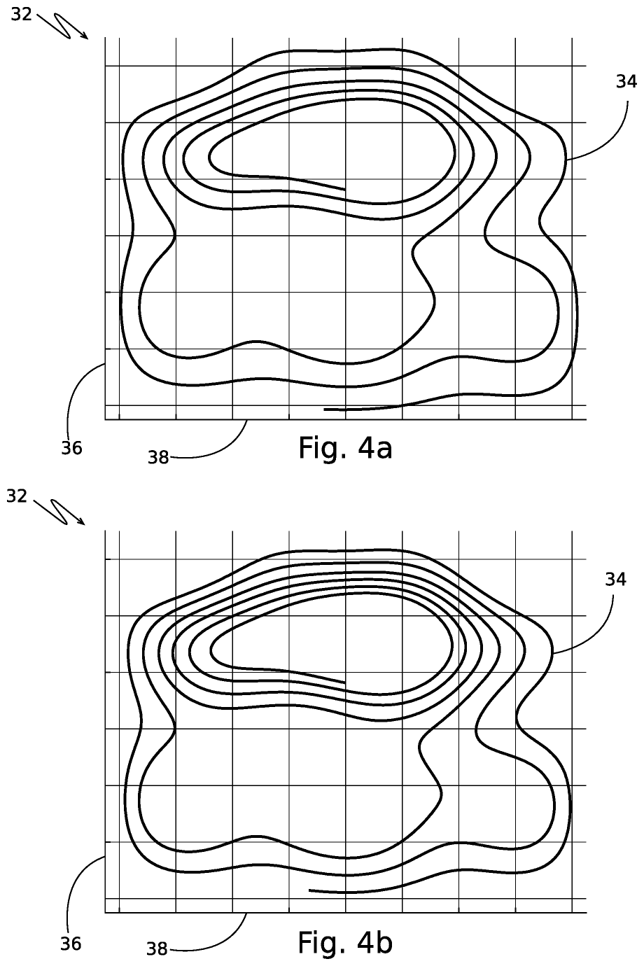

FIG. 2 shows a schematic section through the coil 10 at sectional plane A.

Identical reference signs refer to identical features and are not explained in more detail in FIG. 2 and the following figures.

A conductor cross-section of the electrical cable 12 is the same for all windings in the example shown. It is understood that this is a simplified representation and that different conductor cross-sections can be provided in any case.

Furthermore, it is understood that instead of the planar or flat geometry shown, a curved geometry is also conceivable, which in particular comprises a radius of curvature of at least 60 mm, with a curvature in one dimension less than π/2 rad (90°) arc length or with a curvature in two dimensions on average a solid angle of less than π sr.

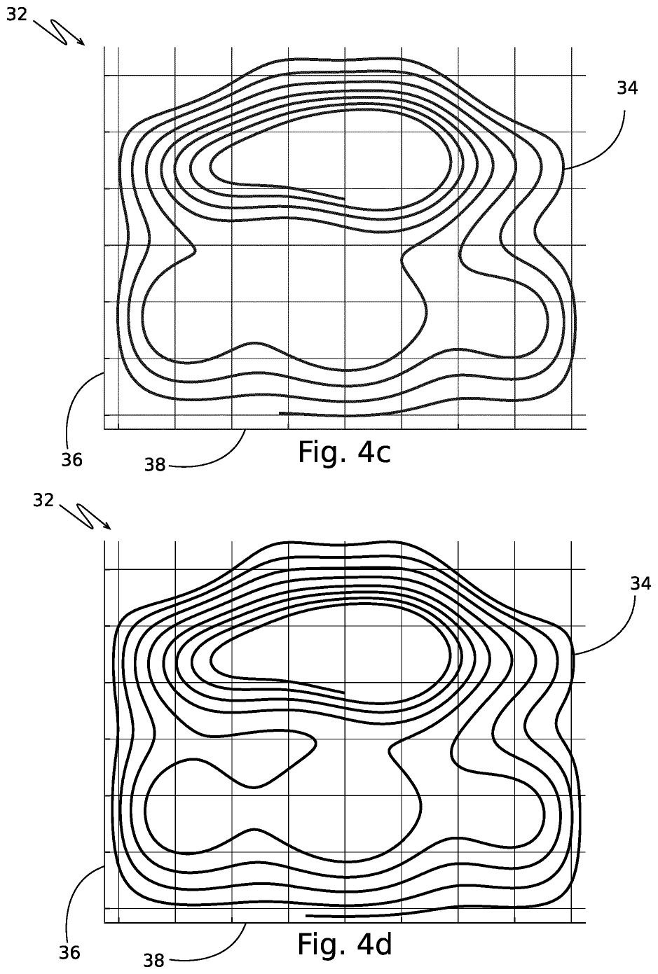

FIG. 3 schematically shows an embodiment example of a system 22 for transcranial magnetic stimulation with a coil 10, in particular a coil according to FIGS. 1 and 2, and a control unit 24.

The control unit 24 is connected to the feed connections of the coil 10 by means of two conductors. It is understood that the supply lines to the feed connections preferably make no or only a negligible contribution to an alternating magnetic field 26 formed by the coil 10. For example, this can be achieved by shielding the feed lines.

The alternating magnetic field 26 of the coil 10 acts on the human brain 28 of a person 30 to be treated.

The control unit 24 is designed to generate an alternating current whose parameters, such as frequency and amplitude, are user-definable. The alternating current fed in by the control unit 24 flows through the electrical conductor 12 of the coil 10.

It is understood that the parameters of the alternating magnetic field 26 can be changed by the parameters of the alternating current flowing through the conductor 12. Furthermore, the parameters of the alternating magnetic field are determined by a geometry of the windings of the electrical conductor 12 of the coil 10.

For reasons of clarity, an insulating structure of the coil 10 is not shown in FIGS. 1 to 3. This insulating structure is arranged between the person 30 to be treated and the surface of the line 12 facing the person 30 to be treated and insulates the line 12 electrically from the person 30 to be treated. This can prevent the person 30 to be treated from suffering an electric shock even if the coil 10 is touched. Furthermore, the mechanical stability of the coil 10 can be increased by the insulating structure.

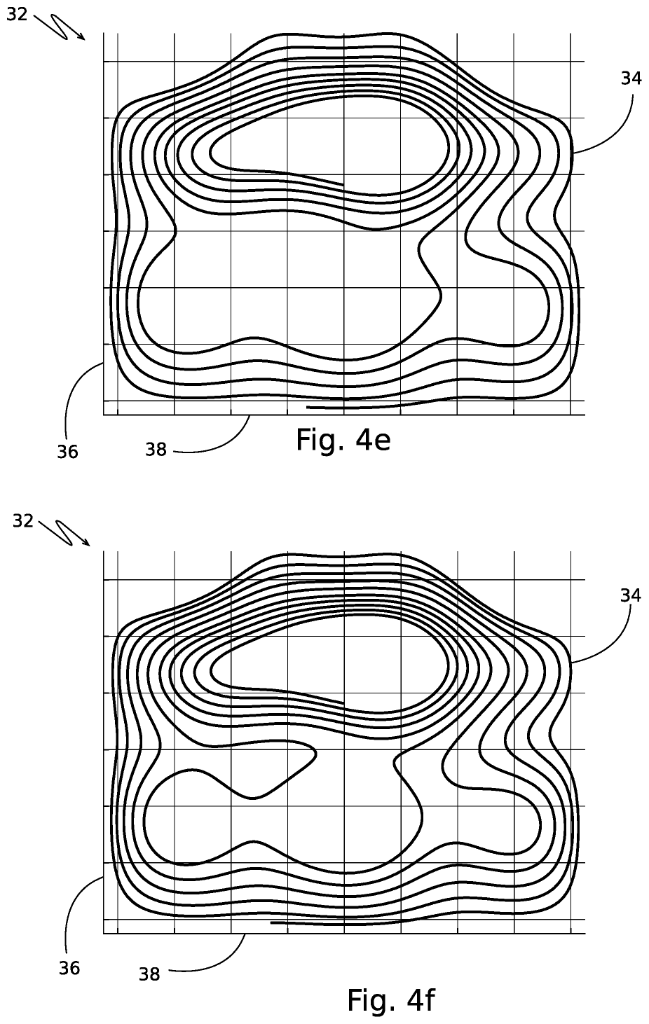

FIGS. 4a to 4i show various courses 34 of the electrical conductor 12 of the coil 10 in top view in diagrams 32.

A Y-axis of the diagrams 32 runs from approx. +80 cm to approx. −40 cm in 20 cm increments. An X-axis runs from approx. −80 cm to approx. +80 cm in 20 cm increments. Each square of a grid shown in diagram 32 for better orientation therefore covers an area of 20 cm×20 cm.

FIG. 4a shows a course 34 with five windings, whereby the first group 18 of inner windings essentially comprises 2.5 windings and the second group 20 of outer windings also comprises approx. 2.5 windings. The winding direction is dominantly parallel. As a result, there are no reverse windings.

The inner windings form an oval, egg-or pear-shaped form. The windings each have a slight indentation on their side with a larger Y value.

The outer windings each have indentations in the X direction approximately in the middle towards the center. In addition, the individual turns of the outer windings have one or two indentations in the negative Y direction.

FIG. 4b shows a course 34 with six windings. This course is similar to the course shown in FIG. 4a, whereby the inner group of windings has an additional winding with a similar course to the other windings in this group.

FIG. 4c shows a further course 34 with seven windings. In contrast to FIG. 4b, the course 34 shown in FIG. 4c has one more outer turn. Furthermore, the indentations of the outer windings in the X direction are more pronounced than in FIG. 4b.

FIG. 4d shows a further course 34 with eight turns. In contrast to the course 34 shown in FIG. 4c, the course 34 shown in FIG. 4d has one more outer turn. The innermost outer winding of the course 34 according to FIG. 4d has very strong indentations in the X direction and Y direction, so that a minimum distance in the X direction between two conductor tracks of the innermost winding of the group of outer windings is approximately 40 cm and a minimum distance between these windings in the Y direction is approximately 20 cm. In an area of this winding surrounding the group of inner windings, the winding has an approximately oval or pear-shaped shape. In an area not enclosing the group of inner windings, the winding has a peanut-like shape.

FIG. 4e shows a further course 34 of an electrical conductor 12. In contrast to the embodiment shown in FIG. 4c, the course 34 shown in FIG. 4e has one more outer winding and one more inner winding. The windings are similar to the course 34 shown in FIG. 4c.

FIGS. 4f to 4i show various courses 34 that are similar to the course 34 shown in FIG. 4d. In these diagrams, courses 34 are shown in which the course 34 comprises one more turn in each case with ascending figure numbering. Here, the number of inner windings and outer windings is alternately increased by 1, whereby the inner windings each have a similar course as in the previously shown figures and the outer windings each have a substantially hexagonal course with indentations in the X and Y directions, whereby the hexagon comprises an approximately square base area with a section drawn upwards on the upper side, i.e. in the negative Y direction. The course 34 shown in FIG. 4f comprises one more outer turn than the course 34 shown in FIG. 4e. The course 34 shown in FIG. 4g comprises one more inner turn than the course 34 shown in FIG. 4f. The course 34 shown in FIG. 4h comprises one more inner turn than the course 34 shown in FIG. 4g. The course 34 shown in FIG. 4h comprises one more inner turn than the course 34 shown in FIG. 4g.

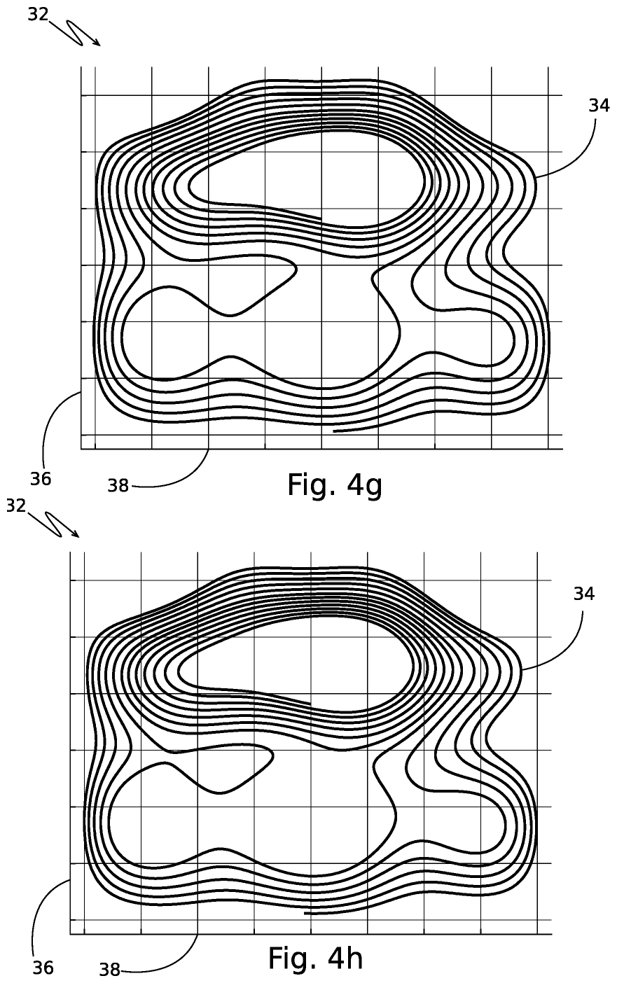

FIG. 5 schematically shows a simulated electric field strength in a human brain 28 normalized. The scale ranges from 0 to 2, whereby the range from 0 to 1 is grayed out and stronger simulated electric fields in limited ranges 40 from 1 to 2 are shown in black and white. Normalized field strengths in the range of 1 are shown in white, normalized field strengths in the range of 2 are shown in black. Normalized field strengths in a range between 1 and 2 in areas 40 are shown in shades of grey.

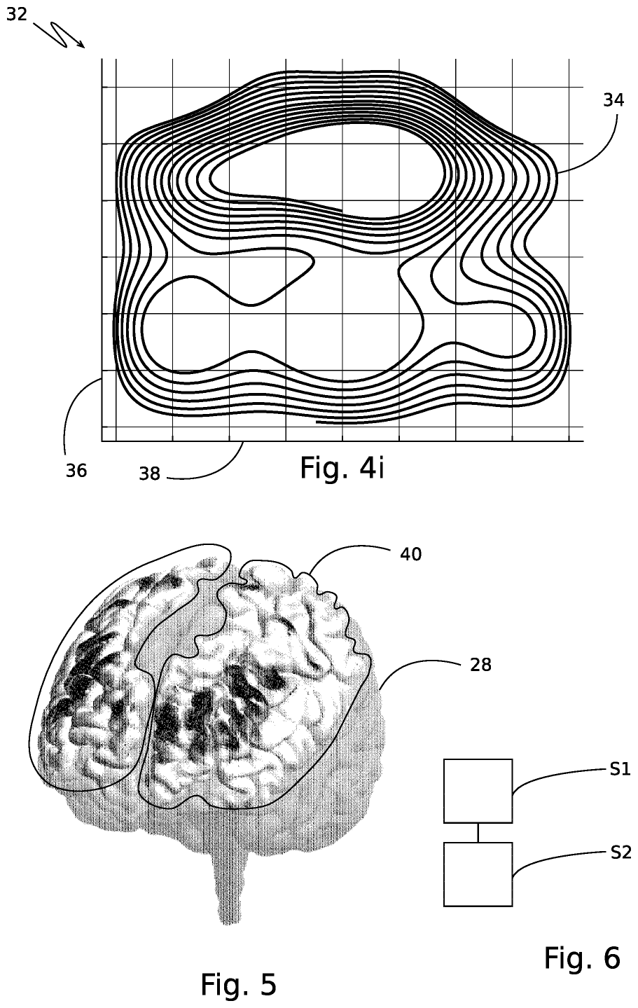

FIG. 6 schematically shows the steps of a method according to the invention for the transcranial magnetic stimulation of a human brain by means of a coil 10 as previously shown in the figures or by means of a system according to FIG. 3.

In a first step S1, the insulation structure of the coil 10 is arranged in an environment of a head of a person 30 to be treated.

In a second step S2, an alternating magnetic field 26 is formed for transcranial magnetic stimulation of the brain 28 of the person to be treated 30 by activating the coil as previously explained with reference to FIG. 3.

The invention has been comprehensively described and explained with reference to the drawings and the description. The description and explanation are to be understood as examples and not limiting. The invention is not limited to the disclosed embodiments. Other embodiments or variations will be apparent to those skilled in the art from the use of the present invention and from a detailed analysis of the drawings, the disclosure and the following claims.

In the claims, the words “comprising” and “with” do not exclude the presence of further elements or steps. The undefined article “one” or “a” does not exclude the presence of a plurality. A single element or unit may perform the functions of more than one of the units recited in the claims. An element, a unit, an apparatus and a system may be partially or fully implemented in hardware and/or in software. The mere mention of some measures in several different dependent patent claims is not to be understood as meaning that a combination of these measures cannot also be used advantageously. Reference signs in the patent claims are not to be understood restrictively.

1. Coi for the transcranial magnetic stimulation of a human brain, which is designed and set up to generate an alternating magnetic field, comprising:
at least one electrical line with a first end and a second end, at each of which a feed connection for feeding the coil with electrical energy is arranged, and which has at least four windings with at least in sections different spacing from the adjacent winding; and
an insulating structure of a non-conductive material for abutment against a head of a person to be treated, which is arranged below the windings of the electrical line in such a way that the insulating structure electrically insulates the line against the head of a person to be treated, wherein
the coil comprises two groups of windings;
a first group comprises inner windings;
a second group comprising outer windings; and
a first surface is formed by a first one enclosing the first group of turns, and a second surface is formed by a second one enclosing the second group of turns; and
the second area is at least 1.5 times as large as the first area.

2. Coil according to claim 1, wherein
the coil is formed with a curvature and is in one dimension on average with less than 2π/3 rad (120°) arc length, preferably less than π/2 rad (90°) arc length and particularly preferably less than π/3 rad (60°) arc length, relative to a center of the curvature or in two dimensions on average with a solid angle of less than π sr (steradian), preferably of less than ⅔ π sr and particularly preferably of less than π/2 sr; and
a radius of curvature of the curvature is at least 60 mm, preferably at least 85 mm and particularly preferably at least 110 mm.

3. Coil according to claim 1, wherein the windings of the coil are arranged planar.

4. Coil according to claim 1, wherein
the windings between the feed connections are arranged without kinks; and/or
comprises at least one winding in at least one area in which the winding has a negative curvature.

5. Coil according to claim 1, wherein
the electrical cable comprises between 4 and 20 windings;
the coil has an inductance in the range from 4 μH to 5 mH, preferably in the range from 5 μH to 500 μH and particularly preferably in the range from 5 μH to 30 μH; and/or
the coil has an electrical resistance in the range from 1 mΩ to max. 1000 mΩ, preferably in the range from 2.5 mΩ to max. 750 mΩ and particularly preferably in the range from 5 mΩ to 50 mΩ.

6. Coil according to claim 1, wherein
the second surface is at least 1.75 times, preferably at least 2 times and particularly preferably at least 2.5 times, as large as the first surface.

7. Coil according to claim 1, wherein
the coil has exactly one single electrical lead forming the windings; and wherein
the windings are preferably arranged without overlapping.

8. Coil according to claim 1, wherein the windings of the electrical line have at least two different centers.

9. Coil according to claim 1, wherein
the windings of the electrical cable have a direction-dependent asymmetry, with:
a first asymmetry with respect to a first direction in coil plane, preferably comprising different conductor densities of the electrical line; and
a second asymmetry with respect to a second direction extending perpendicularly to the first direction in the coil plane, which preferably comprises different bending radii of the electrical line.

10. System for transcranial magnetic stimulation of a human brain, comprising:
a coil according to claim 1; and
a control device for controlling the coil in order to form an alternating magnetic field by means of the coil.

11. A method for transcranial magnetic stimulation of a human brain by means of a coil according to claim 1, comprising the steps of:
arranging the insulating structure of the coil in an environment of a head of a person to be treated; and
forming an alternating magnetic field for transcranial magnetic stimulation of the brain of the person to be treated by activating the coil.

12. A method for transcranial magnetic stimulation of a human brain by means of a system according to claim 10, comprising the steps of:
arranging the insulating structure of the coil in an environment of a head of a person to be treated; and
forming an alternating magnetic field for transcranial magnetic stimulation of the brain of the person to be treated by activating the coil.
